(19) United States
(12) Patent Application Publication
(10) Pub. No.: US 2026/0233160 A1
(43) Pub. Date: Aug. 13, 2026

(54) A SYSTEM FOR GENERATING AND USING NOx GASES IN AN ALGAE GROWTH SYSTEM

(71) Applicant: Paradigm Fuels Pty Ltd, Mount Pleasant, WA (AU)

(72) Inventors: Peter Clifford, Mount Pleasant, WA (AU); Nathan Clifford, Mount Pleasant, WA (AU); David Hack, Mount Pleasant, WA (AU)

(73) Assignee: Paradigm Fuels Pty Ltd, Mount Pleasant, WA (AU)

(21) Appl. No.: 19/154,543

(22) PCT Filed: Feb. 13, 2024
(86) PCT No.: PCT/AU2024/050093

§ 371 Date: Aug. 7, 2025

(30) Foreign Application Priority Data
Feb. 13, 2023 (AU) 2023200794
May 31, 2023 (AU) 2023901706

(51) Int. Cl. B01D 53/84; B01D 21/01; B01D 21/28; C12M 1/00; C12M 1/34

(52) U.S. Cl. CPC B01D 53/84; B01D 21/01; B01D 21/286; C12M 21/02; C12M 21/12; C12M 41/26; B01D 2251/95; B01D 2258/0291

(57) ABSTRACT

A system for generating NOₓ gases for growth of algae comprises: (a) a closed system combustion stage to combust carbonaceous material at a flame temperature and pressure promoting generation of an exhaust gas comprising controlled proportions of carbon dioxide and a determined quantity of NO_{X} gases; (b) a closed system algae growth and oxygen generation stage for receiving the exhaust gas from the closed system combustion stage and delivering said exhaust gas as at least a portion of a feedstock gas for growing algae in an algae growth medium in which solubilised species of nitrogen including at least acid forming NOₓ and its reaction products with algae growth medium are metabolised by growing algae and directing an exit gas comprising carbon dioxide and non-solubilised NOₓ in controlled proportions to the closed system combustion stage; (c) dissociating insoluble NO_{X} received from the closed system algae growth and oxygen generation stage in the closed system combustion stage and reforming said NOx into the same or other NO_{X} gas constituents; (d) creating in situ nitrogen fertiliser in the algae growth medium for the benefit of algae growth; and (e) a control system for controlling operation of the closed system combustion stage and closed system algae growth and oxygen generation stage wherein the control system controls pH of the algae growth medium to achieve a selected condition such as growth of algae or flocculation.

### TECHNICAL FIELD

This invention relates to a system for generating and using NO_{X} gases for use as a nutrient in algal growth and as a flocculation agent to harvest the algae which is used for example in a process for producing a chemical product, in particular a biofuel.

### BACKGROUND ART

The following discussion of the background art is intended to facilitate an understanding of the present invention only. The discussion is not an acknowledgement or admission that any of the material referred to is or was part of the common general knowledge as at the priority date of the application.

All references, including any patents or patent applications, cited in this specification are hereby incorporated by reference. No admission is made that any reference constitutes prior art. The discussion of the references states what their authors assert, and the Applicant reserves the right to challenge the accuracy and pertinence of the cited documents. It will be clearly understood that, although a number of prior art publications may be referred to herein, this reference does not constitute an admission that any of these documents forms part of the common general knowledge in the art, in Australia or in any other country.

NO_{X} gases of which there is a mixture of many constituents (and for example, but not limited to (N₂O, NO, NO₂, N₂O₃, N₂O₄, and N₂O₅) are reasonably well understood with respect to their solubility in water, and also their reaction with water and other gases such as O₂. There is also a great body of literature relating to their formed acids resulting from reactions with water and derived products such as nitrous acid and nitric acid.

Bowman's theory provides an understanding for the calculation of NO gas production and is relevant to exhaust gases from combustion, including from furnaces and combustors (for example as used in gas turbines), and is included by reference: Bowman, C. T. Chemistry of Gaseous Pollutant Formation and Destruction, Chapter 4 of Fossil Fuel Combustion: A Source Book. Bartok, W. and Sarofim, A. F. editors, John Wiley & Sons, Inc., New York, NY, 1991, and clearly defines the relationship between temperature, pressure and Oxygen and Nitrogen gas concentration and the production of NO. This relationship is expressed as:

$$\frac{d [ NO ]}{dt} ( in kgMol / {m}^{3} / sec ) = \frac{1.44 \times {10}^{20}}{{T}^{0.5}} exp {( - 69500 / T ) [ {O}_{2} ]}^{0.5} [ {N}_{2} ]$$

where T is gas temperature in kelvins,
t is reaction time in seconds
   This theory demonstrates that temperature is a driving producer of NO_{X} during combustion of which approximately 95% is NO.

There is literature on the required nitrogen levels required by healthy algae to be supplied by nitrogen fertilising agents, not mentioning the direct use of NO_{X} gases and products of NOx gases, nor demonstrating a method of effectively utilizing insoluble NO_{X} gas components in a closed system and the management of consequential acidification of algae growth medium when NO_{X} gases are efficiently utilised and in which algae are typically grown.

Furthermore, it is accepted that healthy algae requires approximately 5% nitrogen by weight, which places a high commercial impost by way of necessary nitrogen fertiliser in an algae growth system. If for example, urea fertiliser (approximately 47% Nitrogen) was used with costs in excess of USD1000 per tonne, this would imply that 20 ML/year of lipid oil extracted at a yield of 30% from algae requires approximately 3,000 Tons/year of nitrogen (approximately 6,300 tonnes of urea) at a cost in excess of USD6 million/year in fertiliser. This represents a significant and problematic cost of approximately 30% (or more) of revenue from biofuel products derived from lipid processing.

The alternative is to have nitrogen deficiency which affects algal productivity by decreasing photosynthesis, growth rates and longevity. The effects are reported in many studies with the clear implication that considerable nitrogen must be provided to growing algae if lipid oil is to be produced at scale.

International Patent Application Number PCT/AU2022/051155 filing date 27 Sep. 2022, by the present Applicant, and the contents of which are hereby incorporated herein by reference, describe a system and method for growing organisms, in particular algae, which may use NOx as a nutrient, and as an agent for flocculation.

The invention has been developed against the above background.

### SUMMARY OF INVENTION

According to a first broad aspect of the present invention, there is provided a system for generating NO_{X} gases for growth of algae comprising:
(a) a closed system combustion stage to combust carbonaceous material in an oxygen rich gas environment at a flame temperature and pressure promoting generation of an exhaust gas comprising controlled proportions of carbon dioxide and a determined quantity of NOx gases; and
(b) a closed system algae growth and oxygen generation stage for receiving the exhaust gas from the closed system combustion stage and delivering said exhaust gas as at least a portion of a feedstock gas for growing algae in an algae growth medium in which solubilised species of nitrogen including at least acid forming NOx and its reaction products with algae growth medium are metabolised by growing algae and directing an exit gas comprising carbon dioxide and non-solubilised NOx in controlled proportions to the closed system combustion stage; and
(c) dissociating insoluble NO_{X} received from the closed system algae growth and oxygen generation stage in the closed system combustion stage and reforming said NOx into the same or other NO_{X} gas constituents; and
(d) a control system for controlling operation of the closed system combustion stage and closed system algae growth and oxygen generation stage
   wherein the control system controls pH of the algae growth medium to achieve a selected condition. For example, pH may be controlled to a level suitable for growth of algae and flocculation of algae.

In a second broad aspect of the present invention, there is provided a method for generating NOx gases for growth of algae comprising:
(a) combusting carbonaceous material in a closed system combustion stage having an oxygen rich gas environment at a flame temperature and pressure promoting generation of an exhaust gas comprising controlled proportions of carbon dioxide and a determined quantity of NO_{X} gases in a closed system combustion stage; and
(b) receiving the exhaust gas from the closed system combustion stage in a closed system algae growth and oxygen generation stage as at least a portion of a feedstock gas for growing algae in an algae growth medium in which solubilised species of nitrogen including at least acid forming NOx and its reaction products with algae growth medium are metabolised by growing algae and directing a gas comprising oxygen, carbon dioxide and non-solubilised NOx gases in controlled proportions to the closed system combustion stage; and
(c) dissociating under high combustion temperatures, in the closed combustion stage, non-solubilised NOx gases in a closed system combustion stage and reforming into the same or other NOX gas constituents; and
(d) controlling, by a control system, operation of the closed system combustion stage and closed system algae growth and oxygen generation stage
   wherein the control system controls pH of the algae growth medium to achieve a selected condition. For example, pH may be controlled to a level suitable for growth of algae and flocculation of algae.

The term “closed” means stages and/or components and/or sub-systems of the system wherein gasses should not, and desirably cannot, escape outside of the system as a whole.

Carbonaceous material is a material which is rich in, or yields carbon, and for example, plant material and/or bagasse and/or other fuels (such as coal, hydrocarbon gases or other fossil fuels). Carbonaceous material may be selected from a range of fuels with the carbon dioxide and NOx containing stream from a combustor burning the fuel (such as coal, hydrocarbon gases and other fossil fuels) to the “closed algae growth and oxygen generation” stage. This offers an option for scrubbing such streams of carbon monoxide, carbon dioxide, NOx, SOx, mineral, acid, water vapour and other components of furnace off gases using the “closed algae growth and oxygen generation stage”.

Conveniently, as solubilised NOx tends to acidify and lower the pH of the algae growth medium, the control system controls pH of the algae growth medium by mixing the feedstock gas with an alkaline solution and/or alkaline producing gases, to achieve a condition selected from the group consisting of flocculating algae, producing a nitrogen fertiliser and conditioning the pH of the algae growth medium suitable for algae growth.

Composition of the exhaust gas (in particular CO₂ and NOx composition), and correspondingly the feedstock gas delivered to the algae growth medium and in turn the pH of the algae growth medium, is conveniently controlled by controlling a plurality of the following:
(a) concentration of oxygen, CO₂, NOx gases and optionally N₂ directed to the closed system combustion stage;
(b) throughput rate of exit gas from the closed system algae growth and oxygen generation stage to the closed system combustion stage;
(c) combustion temperature;
(d) gas pressure during combustion;
(e) where introduced, throughput rate of nitrogen to the closed system combustion stage, nitrogen preferably being delivered to the closed system combustion stage in minor concentrations in the form of air;
(f) throughput rate of carbonaceous material for the closed system combustion stage; and
(g) quantities and pH of alkaline solution added by the control system.

Preferably, the control system controls pH of the algae growth medium to condition the pH of the algae growth medium suitable for algae growth, optionally prior to the algae growth medium entering the closed system algae growth and oxygen generation stage. The pH may be controlled in a determined acidic range or in a determined alkaline range to flocculate algae, as influenced by the zeta potential of the cell walls of the growing algae species. An advantage of this approach is that the costs of centrifugation for harvesting can be avoided.

A first embodiment comprises flocculation by acidification of algae growth medium. In this regard, the NO_{X} component of the combustion gases may be used with advantage because acidic pH levels are attained in the algae growth medium due to nitric and nitrous acids (as well as other acid gas species: sulphur acids or carbonic acid) formed on contacting of exhaust gas with algae growth medium and/or exhaust vapour/moisture. The acidification of the algae growth medium will, dependent on algae species, overcome Zeta potential on the algae cell walls. A pH of 4 or less (acidic) is desired for algae species of Chlorella vulgaris whereas other algae species will have their own zeta potential and optimum pH (acidic or alkaline) for optimum flocculation.

The introduction of exhaust gases and its components, as described above and including any condensate, to the algae growth medium may further agitate the algae growth medium and force collisions of algae cells to enable binding and flocculation.

The pH of the algae medium, as modified by the formation of nitric and nitrous acids due to combustion stage exhaust gases containing, among other components, soluble NOx aerating the algae medium, is conveniently controlled by controlling combustion stage operation, for example as described above, and algae medium flow rates.

A second embodiment involves flocculation by increasing the pH of the algae growth medium to alkaline conditions with zeta potential for some algae species being overcome under alkaline conditions. Most preferably, pH is increased by introducing an alkalising gas or alkaline solution to algae growth medium containing algae to a level causing flocculation, for example in the pH range 9.5 to 11.

In preferred embodiments, the system comprises a gas production system for producing a gas or alkaline solution formed by dissolution of said gas in water to blend with said feedstock gas within the algae growth medium. Preferably, the gas production system is an ammonia (NH₃) gas or ammonia solution production system and the control system may then control addition of ammonia gas or ammonia solution to the algae growth medium to achieve a determined pH range. Conveniently, the control system controls addition of ammonia and feedstock gas to the algae growth medium to buffer the pH of the algae growth medium. Preferably, nitrogen is primarily supplied to the closed algae growth and oxygen generation stage in the form of ammonia species which would, in particular, include ammonia, ammonia in water and ammonium hydroxide. Nitrogen may also be present in the form of compounds formed by reaction of other soluble nitrogen species, including NOx species and nitrogen based acids, with the ammonia species where introduced to the algae growth medium as in preferred embodiments.

Growth of algae is conveniently achieved in closed growth vessel(s) and flocculation is conveniently conducted in flocculation vessel(s). Each flocculation vessel desirably comprises an agitation means for encouraging flocculation of algae by mechanical agitation or aeration.

Once flocculated, algae from the closed system algae growth and oxygen stage are conveniently collected by a collector, for example by skimming algae from the algae growth medium. Preferably, the collector collects flocculated algae caused to float by agitation and/or aeration.

In embodiments, the system and method conveniently comprise a collector for collecting or harvesting an algae flocculate following:
a. the generation of acids formed from the reactions of gases including NOx gases with water and/or water vapour as introduced to the algae growth medium; or alternatively following
b. the generation of an alkaline solution resulting from the reaction of an alkaline agent—such as an alkaline solution and/or alkaline producing gas—with water as applied to the algae growth medium.

Following flocculation and harvesting, the algae growth medium is desirably conditioned for re-use for growth of algae. In the case of:
a. an acidic algae medium flocculation process utilising nitrogen based acids that are injected into the algae growth medium, the pH of the algae growth medium is desirably conditioned for algae growth by an alkaline solution and/or alkaline producing gas, optionally ammonia or ammonia solution, that is added to the algae growth medium in a conditioning vessel; or
b. an alkaline algae medium flocculation process utilising ammonia gases or ammonia solution injected into the algae growth medium in the flocculation vessel, the pH of the algae growth medium is desirably conditioned for algae growth by nitrogen based acids that are formed in the algae growth medium in a conditioning vessel.

For example, an acid solution formed in the closed flocculation vessel(s), and after flocculation, can be transferred to conditioning vessel(s) and be buffered with an alkaline, for example, ammonia (NH₃), solution which then has the effect of conditioning the pH of the algae medium to a suitable pH for an algae growth environment, in a neutral range for example pH 6.5 to 7.5. The algae growth medium can then be returned to the closed growth vessel(s) containing high levels of nitrogen nutrient for algal growth as provided by both the nitrogen derivatives of NO_{X} from combustion exhaust gases and ammonia compounds, most conveniently from the ammonia production system.

Flocculation and conditioning may, in other embodiments be conducted in the same vessel. Further, any convenient number of flocculation and conditioning vessels may be included within the system.

Both a flocculation vessel and a conditioning vessel used in the system are preferably provided with baffles to form an extended algae growth medium flow path, the baffles forming spaces in which the pH condition of the algae growth medium suited for either purpose, flocculation or conditioning is conducted.

In a flocculation vessel comprising baffles:
I. in the process requiring flocculation in an acidic algae growth medium (as dependent on the algae species), exhaust gas, containing species selected from the group consisting of NO_{X} gases, nitrogen-based acids and other acid forming gases, from the closed system combustion stage is introduced separately to each space; or
II. in the process requiring flocculation in an alkaline algae growth medium (as dependent on the algae species), ammonia is introduced separately to each space
   dependent on pH as monitored in the spaces.

In a conditioning vessel comprising baffles:
I. in the process requiring flocculation in an acidic algae growth medium (as dependent on the algae species), ammonia is introduced separately to each space; or
II. in the process requiring flocculation in an alkaline algae growth medium (as dependent on the algae species), exhaust gas, containing species selected from the group consisting of NO_{X} gases, nitrogen-based acids and other acid forming gases, from the closed system combustion stage is introduced separately to each space
   and dependent on pH as monitored in the spaces.

The baffled conditioning vessel allows progressive pH monitoring and injection into the flow path of the algae growth medium of neutralisation agent, being:
a. ammonia to neutralise an acidic algae growth medium; or
b. exhaust gas containing NO_{X} and/or other acid forming exhaust components, including condensates, from the closed system combustion stage to neutralise an alkaline algae growth medium;
   such that desired pH for algae growth is achieved at least by the outlet of the conditioning vessel.

Further, the conditioning vessel may react nitric acid formed from NOx with ammonia in the algae growth medium to produce in situ a soluble nitrogen fertiliser, such as ammonium nitrate.

The system preferably includes a carbon dioxide and NO_{X} balancing system for balancing carbon dioxide and NO_{X} delivered by exhaust gases from the “closed system combustion” stage with carbon dioxide and NO_{X} requirements in the “closed algae growth and oxygen generation” stage and in particular, for organism growth to produce biomass. Such balance allows efficient use of the carbonaceous material used in the closed combustion stage while maintaining an appropriate physiological response in the algae. The carbon dioxide and NO_{X} balancing system includes a carbon dioxide and NO_{X} storage means.

The closed algae growth and oxygen generation stage preferably at least includes closed growth vessels in the form of “sealed tent(s)”, and more preferably “multi-panelled sealed tent(s)” as described below, for growing waterborne algae, with carbon dioxide and NO_{X} requirements being typically driven by a required carbon dioxide and nitrogen uptake rate of the algae. The “sealed tent(s)” may include—as at least part of the carbon dioxide and NO_{X} balancing system—a carbon dioxide and NO_{X} storage means for storing carbon dioxide and NO_{X} generated by the “closed system combustion” stage, whether in excess of or less than the required carbon dioxide and nitrogen uptake rate of the algae.

The need to store NO_{X} produced by the combustion stage in the gas mix contained in the sealed tent is that not all NO_{X} constituent gases are soluble, and in particular NO and N₂O have low solubility properties, whereas NO₂, N₂O₃, N₂O₄ and N₂O₅ react readily with water to form compounds such as nitric and nitrous acids. Dissolved nitrogen compounds in the algae growth medium operate as a nitrogen nutrient analogous to the way farmers rely on urea which contains approximately 47% nitrogen. The system conveniently enables a proportion of the NO_{X} gases formed in the combustion stage to pass though the closed algae growth and oxygen generation stage (without solubilisation or with re-gasification) to be returned to the combustion stage, and under high heats of combustion and flame temperatures, be dissociated and reformed into the same or other NO_{X} gas constituents.

Dissociating the predominantly insoluble NO component of NO_{X} under high temperature into its nitrogen and oxygen constituents in the combustion stage, and then reforming into the same or other NO_{X} gas constituents provides capacity for, through recirculation, consumption of the NO (typically >90% of NOx in combustion gas dependent on temperature) component of NO_{X}, and environmental management of NO and NOx generally.

A corona discharge device may conveniently be placed in an exhaust stream of the combustion stage and, in the presence of moisture containing gases, will transform significant percentages, for example and dependent on operating conditions, up to about 60% of NO into nitrous and nitric acids with benefit for the process.

A corona discharge device may be placed in the exhaust stream of the combustion stage and, in the presence of moisture containing gases, will also create NOx gases from the nitrogen and oxygen constituents of the exhaust stream gas.

Because flocculation returns generally between for example 50% to 85% algae recovery, remnant algae not flocculated and contained in the algae growth medium that is reintroduced to the closed growth vessel(s) may have been stressed by the acid or alkaline flocculation conditions and be not suitable as algae seed. To address this, fresh seed may advantageously be introduced to compensate for the stressed algae remnant unsuitable for seeding.

Typically, algae are harvested for processing in harvesting and processing stage(s) to produce a further chemical product, in particular from algal lipids as described below. For example, and preferably, algal lipids from the harvested algae may desirably be processed into a biofuel, such as Biodiesel (a fatty acid methyl [or ethyl] ester), Renewable Diesel (a paraffin) and/or other paraffinic fuels such as Sustainable Aviation Fuel (SAF). A range of further chemical products may be produced using carbon dioxide and NO_{X} as a feedstock. Algal lipids are in themselves a chemical product though processing of algae is not limited to processing of their algal lipids, other chemical products present within the algae may also be extracted or further processed to a further chemical product.

The systems described above are advantageously modular. In this way, one or more of the combustion, “algae growth and oxygen generation” stages and associated equipment may be provided as discrete modules which may be replaced with new modules if required to vary capacity, adopt improved technology and/or for maintenance purposes.

The methods and systems described herein enable algal growth for production of chemical products, in particular biofuels, with a significantly lower cost for nitrogen required by growing algae. At the same time, the methods and systems provide the capacity to scrub a range of chemical components from combustion gases—in particular carbon dioxide, NOx and SOx—while providing the ability to produce valuable chemical products.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a block diagram schematically illustrating one embodiment of a system for generating and using carbon dioxide and NO_{X} according to the present invention.

FIG. 2 depicts a schematic long section diagram of a “closed algae growth and oxygen generation” system showing multiple panels within a multi-panelled sealed tent and an acidic algae flocculation configuration and a relationship to other components of the invention and which may be used in accordance with embodiments of the present invention.

FIG. 3 depicts a long section diagram of a vessel that may be used as a closed flocculation vessel or a closed conditioning vessel used to aerate/percolate algae medium with combustion gases containing NOx within systems of embodiments of the present invention.

FIG. 4 depicts a schematic long section diagram of a “closed algae growth and oxygen generation” stage showing multiple panels within a multi-panelled sealed tent and an alkaline algae flocculation configuration and a relationship to other components of the system and which may be used in accordance with embodiments of the system of the present invention.

FIG. 5 depicts a long section diagram of a vessel that may be used as either a closed flocculation vessel or a closed conditioning vessel used to aerate/percolate algae medium with ammonia within systems of embodiments of the present invention.

### DEFINITIONS

The following definitions are provided as general definitions and should in no way limit the scope of the present invention to those terms alone but are put forth for a better understanding of the following description.

Unless defined otherwise, all technical and scientific terms used herein have the same meaning as commonly understood by those of ordinary skill in the art to which the invention belongs. It will be further understood that terms used herein should be interpreted as having a meaning that is consistent with their meaning in the context of this specification and the relevant art and will not be interpreted in an idealized or overly formal sense unless expressly so defined herein. For the purposes of the present invention, additional terms are defined below. Furthermore, all definitions, as defined and used herein, should be understood to control over dictionary definitions, definitions in documents incorporated by reference, and/or ordinary meanings of the defined terms unless there is doubt as to the meaning of a particular term, in which case the common dictionary definition and/or common usage of the term will prevail.

For the purposes of the present invention, the following terms are defined below.

The articles “a” and “an” are used herein to refer to one or to more than one (i.e. to at least one) of the grammatical object of the article. By way of example, “an element” refers to one element or more than one element.

The term “about” is used herein to refer to quantities that vary by as much as 30%, preferably by as much as 20%, and more preferably by as much as 10% to a reference quantity. The use of the word ‘about’ to qualify a number is merely an express indication that the number is not to be construed as a precise value.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

Any one of the terms: “including” or “which includes” or “that includes” as used herein is also an open term that also means including at least the elements/features that follow the term, but not excluding others. Thus, “including” is synonymous with and means “comprising”.

In the claims, as well as in the summary above and the description below, all transitional phrases such as “comprising,” “including,” “carrying,” “having,” “containing,” “involving,” “holding,” “composed of,” and the like are to be understood to be open-ended, i.e., to mean “including but not limited to”. Only the transitional phrases “consisting of” and “consisting essentially of” alone shall be closed or semi-closed transitional phrases, respectively.

Although any methods and materials similar or equivalent to those described herein can be used in the practice or testing of the present invention, preferred methods and materials are described. It will be appreciated that the methods, apparatus and systems described herein may be implemented in a variety of ways and for a variety of purposes. The description here is by way of example only.

As used herein, the term “exemplary” is used in the sense of providing examples, as opposed to indicating quality. That is, an “exemplary embodiment” is an embodiment provided as an example, as opposed to necessarily being an embodiment of exemplary quality for example serving as a desirable model or representing the best of its kind.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The phrase “and/or”, as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

For the purpose of this specification, where method steps are described in sequence, the sequence does not necessarily mean that the steps are to be carried out in chronological order in that sequence, unless there is no other logical manner of interpreting the sequence.

### DESCRIPTION OF PREFERRED EMBODIMENTS

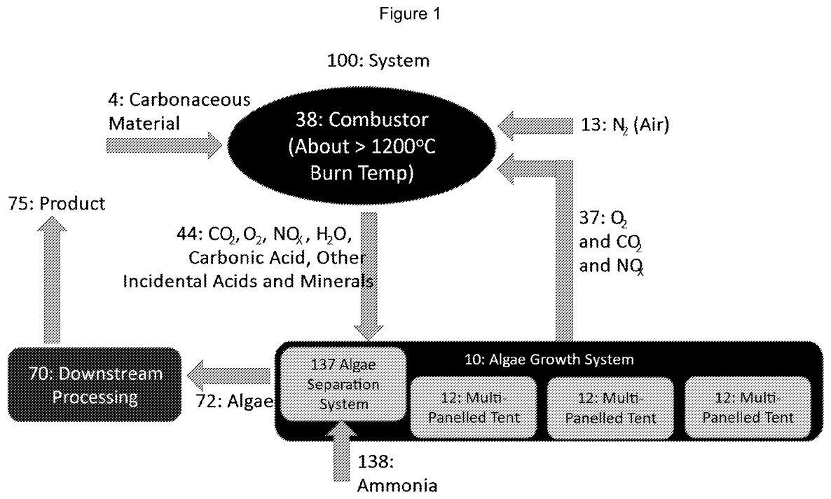

Referring to FIG. 1 there is shown a block diagram of a system 100 for generating and using carbon dioxide and NO_{X} gases to produce a biofuel 75, in particular Biodiesel (a fatty acid methyl [or ethyl] ester), and/or Renewable Diesel (a paraffin) and/or other paraffinic fuels such as Sustainable Aviation Fuel. The system 100 comprises a “closed system combustion” stage 38 containing one or more “closed system combustors or furnace/s” that generates an exhaust gas carbon dioxide, NO_{X} and other gases 44 for consumption in the “closed algae growth and oxygen generation” stage 10. Embodiments include generation of exhaust gas from any combustion process whether or not conducted in a combustor (for example of a gas turbine), furnace or other combustion device.

The closed system combustion stage 38 and closed algae growth and oxygen generation stage 10 co-operate under the control of a control system.

### Closed Combustion Stage

A closed system furnace 38, comprising portion of the “closed system combustion” stage, are preferably is a chain grate and/or fluidised bed and/or blast closed combustion furnace boiler. A plurality of closed system furnaces 38 may be provided. In other embodiments, combustors 38 (for example for gas turbines) may be used rather than furnaces.

Each closed system furnace or combustor 38 may operate with a feed gas of approximately 40% to 90% oxygen and 5% to 50% carbon dioxide and nitrogen containing gas to produce a high concentration of CO₂ and a determined quantity of NOx gases to the “closed algae growth and oxygen generation” stage 10.

The closed system furnace 38 (FIG. 1) is dimensioned to handle the supplied carbonaceous material, and is a closed system preferably intolerant to gas leakage. A chain grate and/or fluidised bed and/or blast closed combustion furnace 38 is preferred in embodiments to combust the carbonaceous material. In other embodiments, combustion may be conducted in a combustor for a gas turbine, for example as used for generation of power at minesites.

The closed system furnace or combustor 38 burns the carbonaceous material 4 in an oxidant gas 37 (as provided by the closed algae growth and oxygen generation system 10) to produce high grade CO₂ and NO_{X} gases 44 that are captured within the closed system furnace or combustor 38 and passed through a heat exchanger for delivery of CO₂ and NO_{X} feedstock 44 to the algae growth and oxygen generation system 10 via the algae separation system 137.

Oxidant gas 37 used for combustion, which contains a mixture of oxygen in major proportion and carbon dioxide in minor proportion and NO_{X} gas, is sourced from the closed algae growth and oxygen generation stage 10. Carbon dioxide and NO_{X} in the gas mixture from the “closed algae growth and oxygen generation” stage is the unconsumed proportion of the CO₂ gas and NO_{X} produced during combustion and oxygen in the mixture and is produced conveniently by the “closed algae growth and oxygen generation” stage, described below.

Air is only preferred in small quantities in combustion due to its high concentration of nitrogen gas which, if not converted to NO_{X}, is a dilutant to the desired high concentrations of oxygen and carbon dioxide and takes up carbon dioxide storage space and consumes gas transfer energy, i.e. energy required for pumping the gas. The preferred “closed system combustion” stage 38 therefore reduces economically detrimental and combustion physics issues which have arisen with use of boiler flue gases as a source of carbon dioxide for algal growth.

The furnace or combustor 38 burns the carbonaceous material 4 in a gas 37 containing high concentrations of oxygen and the remainder carbon dioxide and NO_{X} gases sourced from the “closed algae growth and oxygen generation” stage 10, together with a minor proportion of nitrogen (preferably air) 13. This in-turn enables the production of high concentrations of CO₂ 44 and reconstituted NO_{X} gases to be supplied to the “closed algae growth and oxygen generation” stage 10 and in the case of CO₂ and NO_{X} 44 from the closed system combustion stage 38 after being passed through a heat exchanger to be cooled before supplying to the “closed algae growth and oxygen generation” stage 10. In this regard, exhaust gas from combustion gas is hot and its temperature must be reduced to avoid destruction of algae in “closed algae growth and oxygen generation” stage 10 and more desirably at a temperature within that optimal for algal growth.

In the case of carbonaceous material 4 containing moisture (such as bagasse or wood) the exhaust gases 44 from the closed system combustion stage 38, will also contain condensed water and other condensates and water vapour. In the case of the carbonaceous material 4 being coal, the exhaust gases 44 are likely to contain sulphur containing compounds and minerals commonly found in coal, but lower concentrations of moisture. Furthermore, because there is a likelihood of water contained in plant fuel, such as for example, bagasse, there is the possibility of carbonic acid contained in the combustion exhaust gas formed by the reaction of CO₂ with water under high temperature in the combustion stage. Likewise, if coal was used as a combustion fuel, there is a likelihood of sulphur containing acids and with some carbonic acid. Also present may be other acid gases that will contribute to the acidity of the algae medium when the combustion exhaust gas is percolated through the algae medium in the algae growth and oxygen generation stage as described below. Further, sulphur and other species present in combustion exhaust are typically nutrients that can be utilised in algae growth.

A boiler and/or waste heat to power system is a preferable component of the “closed system combustion” stage 38 and can be used to consume heat generated by a closed system furnace or combustor. A boiler raises boiler fluid temperature, preferably to a steam and at pressure which can be used to drive a steam turbine for the production of electricity. Cogeneration heat obtained from the steam and furnace or combustor of the closed system combustion stage 38 may also be reticulated to feed a heat exchanger, to provide an ability—through provision of heat—to enable other processes in the production of product.

Boiler fluid, prior to it entering the boiler and conversion to steam, can be used as a coolant to establish a multiple stage exhaust gas cooling method suitable to lower gas temperature of the closed system furnace or combustor, for delivery of that gas to the “closed algae growth and oxygen generation” stage at a temperature favourable to algal growth. This method commonly utilises an “economiser” as termed in the boiler industry.

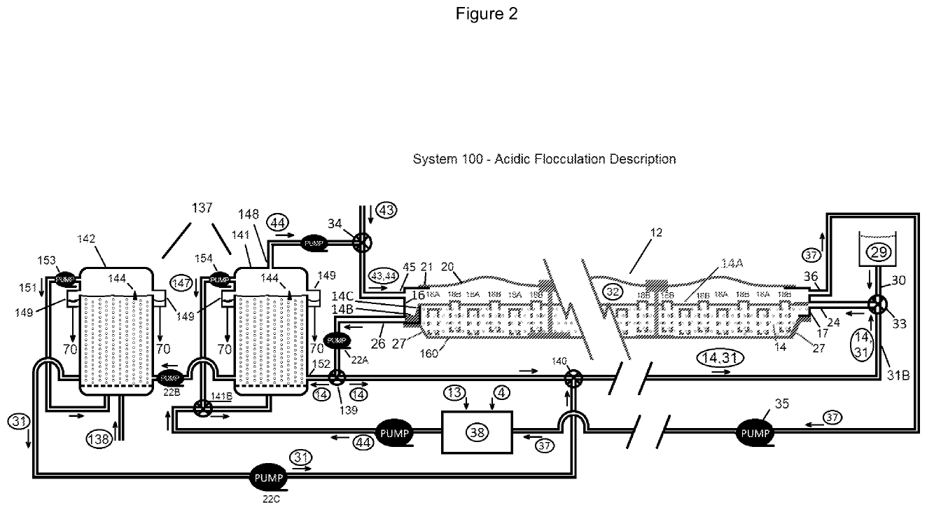

Air is preferably not used as a combustion gas though it may be introduced, as required, as a source of nitrogen to form NOx as determined by the control system and described further below. The closed algae growth and oxygen generation system 10 operates more efficiently using high concentrations of CO₂ feedstock, than gases resulting from the combustion of bagasse 4 in air (containing principally oxygen and nitrogen). With respect to air, and though a small amount of air is required to produce NO_{X}, CO₂ storage within the closed algae growth and oxygen generation system 10 will be adversely impacted if gases containing carbon dioxide and oxygen, but also containing approximately 78% by volume nitrogen, are utilised. That would simply poach space with little benefit to the purpose of the CO₂ and NO_{X} storage facility 32 in FIG. 2.

Carbon dioxide is, in the most part, inert in the closed system combustion stage 38 combustion exhaust gas, though some carbonic acid is likely to be formed, and to a much lesser extent cyanide which undergoes hydrolysis to form ammonia and a small amount of formate. The carbon dioxide passes through that system to enrich the carbon dioxide as produced by the “closed system combustion” stage 38 to be delivered back to the closed algae growth and oxygen generation stage 10. If air was used as a feedstock to the “closed system combustion” stage 38, the nitrogen (78% by volume) would in a lower than preferred temperature environment oxidise to produce some NOₓ gases but would in the main be also inert, both to the closed system combustion stage 38 and the closed algae growth and oxygen generation stage 10. However, by configuring the closed system furnace or combustor 38 to burn at sufficiently high temperatures and with high oxygen concentrations, optionally under pressure, a controlled amount of nitrogen 13 can be introduced to produce NOₓ gas to be processed into a nitrogen fertiliser for the algae medium. Notwithstanding that air (i.e. 78% nitrogen) would, not promote high combustion temperatures and in the main, occupy valuable storage space and is therefore not preferred as a component of the combustion gas of the “closed system combustion” stage 38.

Ultimately, all nitrogen originating from small amounts of air added to the combustion stage (and to a lesser extent, that nitrogen contained in the carbonaceous material used for combustion) will, by reprocessing remnant nitrogen gasses and undissolved NO_{X} by dissociation and recirculation as enabled by the closed system gas communication between the closed algae growth and oxygen generation stage 10 and the closed combustion stage 38, be absorbed by the algae growth medium.

Desirably, the closed system combustion stage 38 operate(s) continuously, preferably year-round, to provide carbon dioxide and NO_{X} as feedstocks to the closed algae growth and oxygen generation stage 10. A consistent or constant feed of carbon dioxide and NO_{X} from the “closed system combustion” stage to the “closed algae growth and oxygen generation” stage is preferable if organism growth is to be maintained at a rate matched with the required production rate of the chemical product, for example a biofuel. The generation of CO₂ and NO_{X} within the closed system combustion stage 38 can be controlled to meet demand by controlling the supply of suitable carbonaceous material as fuel to the closed system combustion stage 38. Burn rate in furnace or combustor may also be controlled with a target set for carbon dioxide production.

The closed system combustion stage 38 can operate at varying burn rates by varying the amount of carbonaceous fuel, enabling flexible and intermittent operation. The carbon dioxide, oxygen and NO_{X} gases balancing system may, as above described, include storage of carbon dioxide, oxygen and NO_{X} gases in case the “closed system combustion” stage generates at any one-time, an imbalance of carbon dioxide and NO_{X} gases against that required to maintain the carbon dioxide and nitrogen uptake rate of the algae, and likewise, the algae producing less oxygen (i.e. at night) than required by the “closed system combustion” stage.

A heat exchanger/s is a component of the closed system combustion stage 38 and is used to remove heat from the CO₂ and NOx containing exhaust gas from that stage 38, suitable for delivery of the gas 44 at desired temperature, as described above, to the closed algae growth and oxygen generation stage 10. Furthermore, heat reticulated as combustion gas or steam from the furnace or combustor 38 can be used via additional heat exchangers to:
a. power a steam turbine electrical generator 190; and/or
b. to preheat algal lipids for downstream processing.

### Closed Algae Growth and Oxygen Generation Stage 10

Closed algae growth and oxygen generation stage 10 here involves growth or cultivation of algae for the purpose of producing biofuel from processed algal lipids. The algae may be any type of waterborne microalgae that requires light energy (e.g. sunlight) for growth. While algae are used in preferred embodiments, it will be appreciated that closed algae growth and oxygen generation stages 10 using alternative, or additional, carbon dioxide respiring organisms or life forms and for producing chemical products other than the production of biofuels are included within the scope of embodiments of the invention.

Algae are grown using a range of nutrients though carbon dioxide and nitrogen species are of particular importance in the described embodiments as alluded to above. Carbon dioxide and NO_{X} gas requirements in the “algae growth and oxygen generation” closed system are typically driven by a required carbon dioxide and nitrogen uptake rate of the algae or other organisms. Carbon dioxide is, as described above, produced by combustion in the combustion stage 38 at substantial concentration, and use for organism growth represents an efficient use of this carbon dioxide. Similarly, because of the high rates of combustion in large amounts of oxygen feed gas, high heats of combustion and flame temperatures allow purposeful and efficient conversion of nitrogen to soluble NOx and other nitrogen species to be efficiently used as nitrogen nutrient in the use of organism growth. In a case where oxygen is produced in the “closed algae growth and oxygen generation” stage, for example by algal respiration, such oxygen—preferably at substantial concentration, for example in the range 40 to 90% by volume—is desirably directed to the “closed system combustion” stage.

Closed algae growth and oxygen generation stage 10 involves one or typically a plurality of vessels, in this embodiment in the form of closed and multi-panelled sealed tents 12, for growing algae, the number of which is determined by CO₂ and nitrogen nutrient supply amongst other factors. These vessels may be termed “closed growth vessels”.

Preferably, each “multi-panelled sealed tent” 12 of the “algae growth and oxygen generation” stage 10 comprises any combination, desirably all, of the following elements:
a. a liquid algae growth medium 14 bearing photosynthetic organisms; and
b. end plates at each end of the “multi-panelled sealed tent(s)” 12, where one end plate(s) 16 (the “far end”) is used to extract liquid algae growth medium bearing organisms which traverse from one end (herein the “near end”) of the “multi-panelled sealed tent(s)” to the other end plate(s) 16 (herein the “far end”); and
c. a supply of seed algae, conveniently through a “near end” plate(s) 17, intended to develop to harvestable concentrations by the time the algae has traversed the length of the “multi-panelled sealed tent(s)” 12, conveniently to the “far end” plate(s) 16; and
d. means for the transfer and/or circulation of water through the “multi-panelled sealed tent(s)” 12, as facilitated by external pumps of the “multi-panelled sealed tent(s)” 12, provides a progression of algae density from seed density at one end to a higher harvestable density at the other end; and
e. a supply of nutrient, and additional water (as for example, algae growth consumes water) which can be added to the transfer and/or circulated water that provides the algae seed density; and
f. construction with flexible impervious translucent material, conveniently in the form of sheet(s) 20 and 160, to form the “multi-panelled sealed tent(s)” 12 and contain the CO₂, oxygen and NO_{X} gases 43,44 stored above the liquid algae growth medium; and
g. with flexible translucent sheets 20, as above described, which conduct sunlight and preferably contain a combination of additives selected from the group consisting of UV protection additives, stabilisers, antioxidants, brightness additives (also called luminescent additives that shift UV to violet and blue spectrum, or alternatively to a red spectrum) and dye (preferably pink dye (i.e. Blueish red) to absorb some of the unwanted light frequencies to provide optimum light spectrum for the growing cycle of algae); and
h. a source of carbon dioxide (CO₂) and NO_{X} gases 43,44 to be delivered—desirably through, or close to, one end plate—and intended to occupy the CO₂, oxygen and NO_{X} gases storage space 32 above the liquid algae growth medium with Henry's Law and the high solubility of CO₂ and some constituent gases of NO_{X} in water ensuring the mass transfer of CO₂ and constituent gases of NO_{X} from the CO₂, oxygen and NO_{X} gases storage space 32 by diffusion into the liquid algae growth medium 14 enriching it with CO₂ and nitrogen, to cultivate the algae; and
i. a gas offtake, in or near the other end plate(s) (that is, the other end of the “multi-panelled sealed tent(s)” from which the CO₂ is delivered) from which undissolved NO_{X} and oxygen/carbon dioxide gas mix as generated by the algae, can be bled from the CO₂, oxygen and NO_{X} gases storage space 32 of the or each “multi-panelled sealed tent(s)” 12 to return that gas mix to the “closed system combustion” stage 38 or be vented; and
j. external to the or each “multi-panelled sealed tent(s)” 12, pump(s) 22A, 22B and 22C and algae separation system(s) 137 fitted to maintain a circulation of nitrogen enriched liquid algae growth medium 14,31 (with seed organisms) through “multi-panelled sealed tent(s)” 12, desirably from the “far end” plate 16 and algae separation system(s) 137 back to the “near end” plate 17; and
k. matter or nutrients promoting organism growth to be provided to organisms borne by the liquid algae growth medium 14, conveniently as supplied through the “near end” plate 17; and
l. optionally, a layer of ballast, such as steam sterilised soil, on the floor of the “multi-panelled sealed tent(s)” 12 to provide ballast and stability in times of high wind, and provide thermal ballast to assist in maintaining a more constant water temperature; and
m. a sealed algae separation system(s) 137 comprising a closed flocculation vessel(s) 141 communicating with a closed conditioning vessel(s) 142 for each or shared amongst multi-panelled sealed tent(s) 12 to agitate and flocculate liquid algae growth medium 14 bearing organisms extracted from the far end of the multi-panelled sealed tent(s) 12. Agitation to cause algae to collide and flocculate in the algae medium is preferably achieved by aeration using recycled gases 147,151 within each vessel(s) or those gases obtained from the closed combustion stage 44 or aerated ammonia 138; and
n. optional mechanical agitation is also provided for; and
o. the aeration of gases to allow the flocculated algae to float and be skimmed off the surface of the algae medium within the closed vessel(s) of the sealed algae separation system(s) 137; and
p. the arrangement and functionality of vessels within the algae separation system; and
q. the carbon dioxide, oxygen and NOx gases from the combustion stage, once percolated through the closed flocculation or conditioning vessel(s), are delivered by manifold to the multi-panelled sealed tent(s) 12, preferably at the far end, to allow a more controlled management of the pH levels of the algae medium within the multi-panelled sealed tent(s) 12, and to supply higher concentrations of carbon dioxide to a denser algae solution, which occurs at the far end, compared to that algae density at the near end.

Closed algae growth and oxygen generation stage 10 is controlled using sensors and control systems (for example, the same SCADA control system as used to control system 100) to monitor and control preferably, but not limited to, a combination of any, or all, of the following:
a. depth of the water;
b. the temperature of the liquid algae growth medium;
c. the pressure of gas in the “multi-panelled sealed tent(s)” and sealed algae separation system(s);
d. pH levels of the liquid algae growth medium at different stages of the process;
e. valves to manage the CO₂ and NO_{X} flow through the sealed algae separation system(s) and panels within the “multi-panelled sealed tent(s)”;
f. CO₂ and NO_{X} gas concentrations and flow rates;
g. the O₂/CO₂ extraction from the “multi-panelled sealed tent(s)”;
h. the top-up water/nutrient supply;
i. ammonia delivery rates;
j. algae medium flow rates;

The number of multi-panelled sealed tent(s) 12 that may be deployed in the system is selected dependent on factors such as the amount of carbonaceous material combusted (and hence CO₂ and NO_{X} generated in the “closed system combustion” stage 38 and thence CO₂ and nitrogen compounds directed to the multi-panelled sealed tent(s) 12 for metabolism by growing algae).

Carbon dioxide and NO_{X} gases are, in embodiments, captured from the closed system combustion stage 38 and transferred to the algae separation system 137 where acidic nitrogen compounds or species are formed from the NO_{X} and ultimately the gases are transferred to the “multi-panelled sealed tent(s)” 12 through a pressure differential system that pumps carbon dioxide, residual oxygen and NO_{X} gases from the “closed system combustion” stage 38.

During growth of algae, oxygen is produced through algal photosynthesis. Thus, oxygen containing gas 37, to which algally respired oxygen is added, is trapped under the seal or ceiling 20 of the “multi-panelled sealed tent” 12 (refer to FIG. 2) and may be collected at a gas offtake 36 to function as the oxygen supply 37 and delivered by pump 35 to the “closed system combustion” stage 38. This oxygen 37 will contain CO₂ and NO_{X}, by virtue of the storage facility 32 also containing CO₂ and NO_{X}.

The oxygen containing gas 37, being in the main for reasons described above, a mixture of high grade oxygen and the remainder carbon dioxide and NO_{X}, is buffered within the “multi-panelled sealed tent” 12 in the CO₂ storage facility 32 (FIG. 2) in that the CO₂ storage facility space 32 can be dimensioned to store several days supply of CO₂ and undissolved NO_{X} in the presence of for example about 60% oxygen by volume to be directed to the “closed system combustion stage” 38. This allows the “closed system combustion” stage 38 to operate at a burn rate that may be matched with the average CO₂ 44 and nitrogen uptake rate of the waterborne algae in the algae medium 14.

Without wishing to be bound by theory, in the furnace(s) or combustor(s) of the closed system combustion stage 38, the combustion of a carbonaceous material such as coal generates approximately 1 mole of CO₂ to 1 mole of O₂ consumed. Similarly, in the case of carbonaceous material being cellulose and lignins there is also an equivalent molar balance of about 1 mole CO₂ produced to 1 mole of O₂ consumed. Then if a molecular balance of lipid and carbohydrate component creation is considered in the process which occurs in algae growth within the closed algae growth and oxygen generation stage 10, there is a very approximate equivalent amount of 130 moles of oxygen released by the algae to 117 moles of CO₂ consumed by the algae.

In the balance, there is not as much oxygen required to produce CO₂ in the “closed system combustion” stage 38 as oxygen produced by the “closed algae growth and oxygen generation” stage 10 and there is likely to be an oxygen surplus, enabling the high oxygen concentration of feed gases to the closed system combustion stage.

One of the forms of NO_{X} is NO₂ which reacts with water to form nitric and nitrous acids, and typically represents about 5% of the NO_{X} gases. Two significant pathways are considered herein, though this does not exclude other pathways and other constituents of NO_{X}, which are provided by way of example:

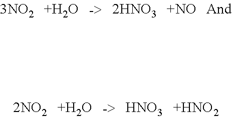

The major constituent gas of NO_{X} from combustion is NO (and about >90% concentration of the NOx) which is not very soluble and is inefficient in providing nitrogen into the algae medium. Thus, it is the approximate 5% NO₂ component of NO_{X} that is suitable in the systems and methods described herein.

A large number of “multi-panelled sealed tents” 12, potentially many hundreds of “multi-panelled sealed tents” 12 could be included dependent on factors such as biofuel production targets and/or exhaust gas output from a power station where the system 100 is used to scrub NOx and other acid gases from that exhaust gas.

A cross section of a panel contained in an algae “multi-panelled sealed tent” 12 is shown in FIG. 2 and described, for purposes of exemplification, below. Growth of algae in “closed algae growth and oxygen generation” stage 10 requires light energy, carbon dioxide, nutrients (in particular those comprised in combustion gas from combustion stage 38 and which include NOx and SOx dependent on the content of sulphur in the carbonaceous material combusted in combustion stage 38) and a growth medium.

Each “multi-panelled sealed tent” 12 comprises a floor 160 and translucent cover (seal or ceiling 20). Each “sealed tent(s)” 12 should be water and gas-tight during algal growth and is desirably at least partially inflatable to accommodate differing volumes of carbon dioxide, oxygen and NO_{X}. Each “multi-panelled sealed tent” is terminated at each end with respective end plates 16, 17. The “far end” plate 16 effects the removal of liquid algae growth medium 14 from the “multi-panelled sealed tent” 12 and also the supply of an oxygen/carbon dioxide and NO_{X} gas mixture 43,44 into the “multi-panelled sealed tent” 12 and the “near end” plate 17 effects the removal of oxygen, carbon dioxide and NO_{X} gas 37 from the “multi-panelled sealed tent” 12 and also the supply of liquid algae growth medium 14,31 into the “multi-panelled sealed tent” 12.

A “multi-panelled sealed tent” 12 for the purpose of growing algae, contains an algae growth medium 14 comprising a liquid suitable for supporting algal growth. In particular, the growth medium 14 comprises water which is contained in and constrained by the “multi-panelled sealed tent” 12, having a surface water level 14A within which waterborne algae flow (i.e. move) from a “near end” plate 17 of the “multi-panelled sealed tent” 12 to the “far” end plate 16. The “multi-panelled sealed tents” 12 may be located within a construction or excavation, for example, to provide a supporting structure and reduce the height of the “multi-panelled sealed tent” above ground.

The “multi-panelled sealed tent” 12 is a closed system in which algae are grown in isolation from airborne pollutants and stray algal cells. There is no apparent limitation on algae that may be grown in the system. Chlorella vulgaris is well known algae which may be adopted in preferred embodiments. As described in US Patent Publication 20210079338, other species that may be grown include unicellular and multicellular algae. Such algae may include rhodophytes, chlorophytes, heterokontophytes, tribophytes, glaucophytes, chlorarachniophytes, euglenoids, haptophytes, cryptomonads, dinoflagellum, phytoplankton and the like and combinations thereof. Algae may be of the classes Chlorophyceae and/or Haptophyta. Suitable microalgae may include one or more of the following species: Achnanthes, Amphiprora, Amphra, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Ellipsoidon, Emiliania, Eremosphaera, Ernodesmius, Euglena, Franceia, Fragilaria, Gloeothamnion, Haemotococcus, Haloacafeteria, Hymenomonas, Isochrysis, Lepocinclis, Micractinium, Monoraphidium, Nanochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus, Pichochlorum, Pseudoneochloris, Pseudostaurastrum, Platymonas, Pleurococcus, Prototheca, Pseudochlorella, Pyramimonas, Pyrobotrys, Scenedesmus, Schizochlamydella, Skeletonema, Spyrogyra, Stichococcus, Tetrachlorella, Tetraselmis, Thalassiosira, Tribonema, Vaucheria, Viridiella, and Volvox species.

Closed algae growth and oxygen generation stage 10 further comprises a transparent seal or ceiling 20 for closing and sealing the “multi-panelled sealed tent” 12; pumps 22A, 22B and 22C for moving the liquid algae growth medium 14 bearing algae throughout the “multi-panelled sealed tent” 12 and algae separation system 137; and an inlet 24 for recirculating liquid algae growth medium 14, replacing water consumed by algae and/or lost to the process, injecting or otherwise delivering or introducing matter, such as nutrients, promoting algal growth through the “near end” plate 17 of the “multi-panelled sealed tent” 12.

The material of the transparent or translucent seal 20—and any other portions through which sunlight is to travel—desirably include UV stabilisers and other chemical additives to constrain the wavelength of light transmitted into the “multi-panelled sealed tent” 12. For example, the green portion of the light spectrum does not deliver light conducive to algal growth so an additive such as a dye (desirably pink in colour) may be used to exclude the green portion of the visible light spectrum.

Constructional details of seal 20 and structure of system 100 are described in the Applicant's International Patent Application No. PCT/AU2022/051155 incorporated herein by reference and are not repeated here.

If, in the example cited in the Applicant's International Patent Application No. PCT/AU2022/051155, the movement of algae is controlled with an inflow of liquid medium 14 into the “multi-panelled sealed tent” 12 through the inlet 24 of approximately 15 m³/hr and a similar outflow at the outlet 26, it will take 16 days to traverse the “multi-panelled sealed tent” 12 (the life span of algae is about that duration).

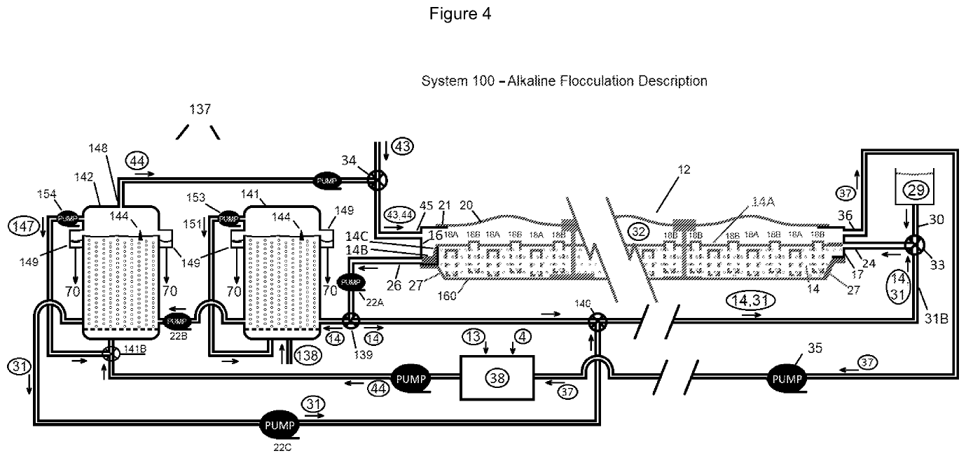

With reference to FIGS. 2 and 4 liquid algae growth medium 14 promoting organism growth in the multi-panelled sealed tent 12, is injected into the multi-panelled sealed tent 12 via an inlet pipe 24 fixed to the “near end” plate 17. The inlet pipe(s) 24 can be used to:
a. recirculate liquid algae growth medium 14 as recirculated liquid algae growth medium 31; and/or
b. recirculate liquid algae growth medium 14 containing unprocessed algae seed that has bypassed the algae separation system 137 the amount of which is controlled by a valve(s) 139; and/or
c. replace water consumed by algae and/or lost to the process with top-up water from reservoir(s) 29; and/or
d. inject or otherwise deliver or introduce matter such as nutrients in the recirculated liquid algae growth medium 31 and/or via reservoir(s) 29; and/or
e. introduce algae seed to promote algal growth via recirculation pipe 31B and/or reservoir(s) 29;
   the source and amount of which, when that source is divisible to one or more reservoir(s) 29, is controlled by valve(s) 33.

In this embodiment, inlet pipe 24 is operable to inject, or otherwise feed, introduce or deliver, matter into the “multi-panelled sealed tent” 12 via an injection, or feed/delivery via pipe 30 and/or pipe 31B (which are controlled by a valve(s) 33) to supply the inlet pipe 24.

It may be appreciated that nutrient matter may be injected through the inlet pipe 24, though additional injector(s) may be provided if required. For example, in embodiments, matter such as one or more nutrients suitable for the algae being grown, may be injected into the algae growth medium 14. An algae growth medium as known in the art is suitable for provision of such nutrients other than the nutrients as described herein.

The inlet pipe(s) 24 directs the liquid algae growth medium 14 through the “near end” plate 17 of the “multi-panelled sealed tent” 12 at an injection rate commensurate with the desired algae density profile and water depth 14A required over the length of the “multi-panelled sealed tent” 12.

### Ammonia Production

Ammonia, when mixed with water as present in algae growth medium 14 is an alkaline solution suitable as a flocculation agent and as a fertiliser and is used in preferred embodiments to buffer acids formed by aeration of algae growth medium 14 with combustion stage 38 exhaust gases in the closed conditioning vessel(s) 142 to establish a healthy algae medium for injection back into the “multi-panelled sealed tent(s)” 12. The benefit of ammonia which in itself is highly soluble, is that because of its high nitrogen content (approximately 82% by weight), it is particularly suitable for making up any shortfall between available nitrogen in dissolved NOx gases and ammonium nitrate and nitrogen necessary in the algae growth medium. Thus, system 100 conveniently includes an ammonia production system or plant (not shown) in which ammonia is produced, for example, by the Haber process. The ammonia production plant may, as an alternative to ammonia gas, provide a solution of ammonia in water for use in flocculation and conditioning steps involving closed flocculation and conditioning vessel(s) 141, 142.

In the following descriptions “both vessel(s)” or “respective vessel(s)” refers to both the closed flocculation vessel(s) 141 and the closed conditioning vessel(s) 142.

### Algae Separation System 137

Liquid algae growth medium 14 is delivered to the “Algae Separation System(s)” 137 via outlet pipe 26 and pumped via pump 22A to a diversion valve 139 and then to flocculation for algae separation through collection or harvesting of algae. Flocculation provides a cost effective alternative to collection or harvesting by centrifugation. In preferred embodiments, flocculation is achieved by the control system controlling pH in a determined acidic range or in a determined alkaline range to flocculate algae, the choice of which range is influenced by the zeta potential of the cell walls of the growing algae species. An advantage of this approach is that the costs of centrifugation for harvesting can be avoided.

Flocculation is described in Maji G K et al. Microalgae Harvesting via Flocculation: Impact of PH, Algae Species and Biomass Concentration. Methods Microbiol Mol Biol. 2018 April; 1(2): 106, the contents of which are hereby incorporated herein by reference. Without wishing to be bound by theory, the surface charge (zeta potential) of microalgal cells determines flocculation nature of the biomass. When the zeta potential is high (>25 mV, positive or negative), electrical repulsion between particles is strong and when it is close to zero, particles can approach each other to a point where they will be attracted by Van der Waals forces. When that happens, algae will aggregate and flocculation will occur.

Algae medium 14 is directed via a diversion valve 139 to the flocculation vessel 141 as demonstrated for the acidic flocculation embodiment of FIG. 2 or alternatively the alkaline flocculation embodiment of FIG. 4.

In both flocculation embodiments, the algae medium is transferred from the closed flocculation vessel(s) 141 to the closed conditioning vessel(s) 142 in which the pH of the algae medium is conditioned suitable for algae growth and return to the multi-panelled sealed tent 12.

In both flocculation embodiments, the gases 44 from the combustion stage 38 are used to aerate the algae medium 14 contained in either:
a. the closed flocculation vessel(s) 141 in the case of acidic flocculation or
b. the closed conditioning vessel(s) 142 in the case of alkaline flocculation
   and which are herein referred to as the closed respective vessel(s) 141 or 142 depending on the above pH of the flocculation. In the closed respective vessel(s) 141 or 142 acid forming gases from exhaust gas 44 solubilise to form aqueous acids as described below. The undissolved components of the exhaust gas 44 are then captured and removed via outlet pipe 148 from the enclosed space between water level of the algae growth medium 14 and the ceiling of the above closed respective vessel(s) 141 or 142 and redirected, via mixing valve 34, to the multi-panelled sealed tent 12 via injector 45 as gaseous nutrient for algae growth and to maintain levels of CO₂ in the CO₂ and NO_{X} storage facility 32, which forms the means for storing carbon dioxide and NO_{X}.

In both embodiments, additional CO₂ gas 43 may mix with, and complement, the gas mixture of 44 following introduction of additional CO₂ gas 43 through mixing valve 34. Such additional CO₂ gas 43 may be obtained from parallel processes such as fermentation of sugar containing plant material, in which case bagasse is a preferred carbonaceous feedstock 4 to the furnace 38.

Carbon dioxide, NO_{X}, carbonic acid, other incidental acids, minerals and remnant oxygen gases 44 sourced from the closed system combustion stage 38 are directed via a manifold to the above closed respective vessel(s) 141 or 142 of the algae separation system 137 via valve 141B controlled by the control system. Valve 141B controls the inflow of gases 44 into the above closed respective vessel(s) 141 or 142 as well as mixing recirculated gases 147 pumped 154 from the top of the above closed respective vessel(s) 141 or 142 to assist in the consumption of all acid forming gases in the contained algae growth medium 14 and provide a desired pH therefor.

Aeration may include recycled gases captured in the closed space between algae medium and the ceiling of the vessel(s) 141 or 142 and pumped back through the aeration system to effectively use all highly soluble gases.

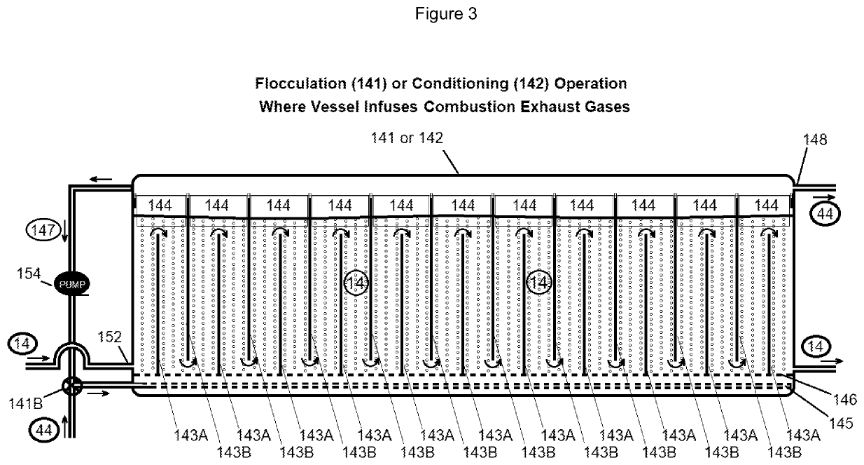

The feed streams of gases 44 and 147 into the closed respective vessel(s) 141 or 142 are reticulated in a reticulation system 145 (FIG. 3) below the aeration base plate 146 to assist in uniformity of gas supply and aeration across the length of the closed respective vessel(s) 141 or 142.

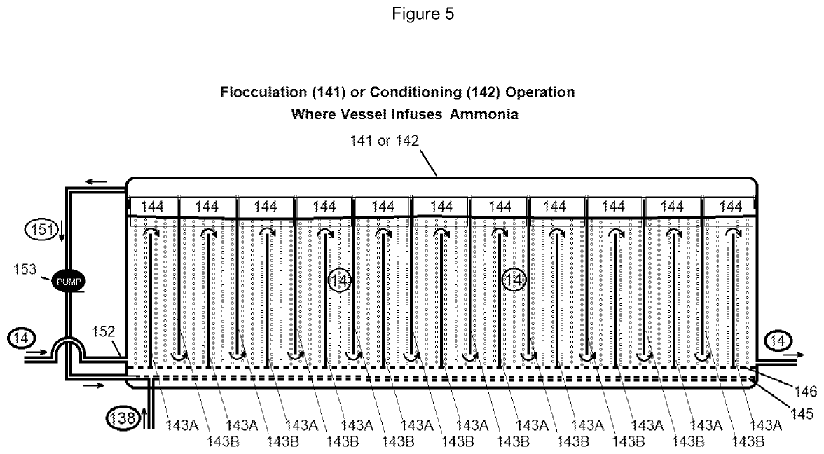

Similarly, the feed of recirculated ammonia gases 151 (FIG. 5) into the
a. closed flocculation vessel(s) 141 in the case of alkaline flocculation; or
b. the closed conditioning vessel(s) 142 in the case of acidic flocculation are reticulated in a reticulation system 145 below the aeration base plate 146 to assist in uniformity of gas supply and aeration across the length of the closed respective vessel(s) 141 or 142 and aerate the ammonia infusion.

Ammonia 138 is preferably separately reticulated in its reticulation system 145 (FIG. 5) in the closed respective vessel(s) 141 or 142, as above described, to assist in the delivery of ammonia at points along the length of the same closed respective vessel(s) 141 or 142 dependent on monitored pH.

In the embodiments shown, ammonia 138 or exhaust 44 from combustion stage 38 is delivered into spaces between baffles 143A and 143B laterally spaced along vessels 141 and 142.

Algae medium is injected 152 into both vessel(s) 141 and 142 and along a protracted route over baffles 143A that are attached to the aeration base plate 146 (which stops any underflow of algae medium 14 below baffles 143A) and the algae medium then flows under baffles 143B. Baffles 143B project above the algae medium water level to inhibit the flow of algae medium across the tops of baffles 143B.

Aeration of gases 44 and 147 and aeration of gases 151 and ammonia species (in particular NH₃, NH₃ in water and ammonium hydroxide) 138 in the flocculation vessel 141 and conditioning vessel 142 as dependent on the configuration for the pH type of flocculation, through algae medium 14 cause agitation, to allow algae cells to collide and flocculate and cause the flocculate to float to the surface of the algae medium surface, where the flocculate may be collected, for example by skimming. Mechanical agitation may also be applied to the flow of algae medium between baffles 143A and 143B by installing propellers between the baffle walls to mix the algae medium 14. Further, the number of baffles 143A and 143B is selected to effect sufficient algae medium flow rate and travel time within the vessel(s) to achieve flocculation, which could take by way of example a minimum of 15 minutes.

Propellers may also be included to overcome head loss between baffles 143A and 143B longitudinally along the flocculation vessel(s) 141 and/or conditioning vessel(s) 142 and maintain a constant water level to aid the skimming of flocculated algae into a spill tray 149 located either side of vessels 141 and/or 142.

Irrespective of where the majority of the flocculation occurs (either the closed flocculation vessel(s) 141 or the closed conditioning vessel(s) 142) both vessel(s) are desirably enabled for aeration and skimming. Substantially the majority of flocculation occurs in the flocculation vessel(s) 141 but will invariably occur in part by force of aeration in the conditioning vessel(s) 142.

Aeration of gases 44 and 147 and aeration with gases 151 and ammonia species 138 in respective vessel(s) 141 or 142 enables the flocculated algae to float to the surface of the algae growth medium 14 contained within both vessel(s) 141 and 142 where it can be collected.

Collectors, in the form of skimmers 144, are installed in both vessel(s) 141 and 142 on and between each projecting baffle 143B, to move the flocculated algae laterally across the vessel(s) 141 and 142 to be captured in a spill tray 149 suitably located at the water level within the vessels(s) 141 and 142 of the algae growth medium 14.

The skimmers 144 are conveniently mounted on rollers that run along the top of the baffles 143B and are winched laterally across both vessel(s) 141 and 142 to skim algae from the algae growth medium 14.

A small portion of the algae growth medium 14 is redirected back through the diversion valve 139 to the near end of the multi paneled sealed tent 12 via valves 140 and 33 using recirculation pipe 31B, to provide unprocessed algae seed that has bypassed the algae separation system 137 and the inherent destructive action of the acid and alkaline flocculation processes on the algae that is processed.

In both embodiments, whether relating to acidic flocculation or alkaline flocculation, flocculation relies on overcoming the above described Zeta potential of the algae cell walls and on agitation to force collisions of algae cells to form a flocculate.

### Acidic Flocculation

In the acidic flocculation embodiment, a pH of 4 or less (acidic) is desired for algae species of Chlorella vulgaris though other algae species will have their own zeta potential and optimum pH (acidic or alkaline) for optimum flocculation. A pH lower than pH 4.0 is a preferable pH range to flocculate some algae species such as, for example, Chlorella vulgaris algae. At a pH of 3.5 Chlorella vulgaris exhibits approximately 75% flocculation efficiency.

Flocculation vessel(s) 141 will both acidify the algae medium (thereby overcoming Zeta potential on the algae cell walls, as described above), and further agitate the algae medium and force collisions of algae cells to enable binding and flocculation and furthermore, aerate the flocculate for removal using for example, skimming the algae medium surface laterally as described above.

The spent algae medium would preferably be removed from the base of the closed flocculation vessel(s) into a closed conditioning vessel(s), where it can be neutralised with ammonia. Once neutralised it is returned to the end of the multi-panelled sealed tent 12 where algae seed is introduced.

### Alkaline Flocculation

In the alkaline flocculation embodiment, the majority of the algal flocculation occurs in the flocculation vessel 141 aerated with ammonia gases and/or infused with ammonia solution and the spent alkaline algae growth medium is transferred to the closed conditioning vessel 142 via pump 22B where combustion gases containing NOₓ and acids are percolated through the algae medium 14 to neutralise the alkaline solution, as described above.

The closed ammonia infused flocculation vessel(s) 141 have similarities in construction to the closed conditioning vessel(s) 142 as schematically shown in FIG. 5. Ammonia 138 is delivered to the closed ammonia infused flocculation vessel(s) 141 where it is mixed with the algae growth medium 14 to make it alkaline, for example pH range of approximately 9 to 11.5 though the preferred pH is influenced by the zeta potential of the algae species grown in the closed system algae growth and oxygen generation stage 10.

The algae growth medium 14 contained within the closed ammonia infused flocculation vessel(s) 141 is aerated preferably with recirculated gases 151 captured from the closed space or void between the surface of the algae medium 14 and the ceiling of the closed ammonia infused flocculation vessel(s) 141. Pump 153 provides the necessary pressure and gas flow to achieve the desired level of agitation in the algae growth medium 14.

Fouling of the closed ammonia infused flocculation vessel(s) 141 is minimised by removing aerated flocculate, for example by skimming the algae medium surface laterally as described above.

The flocculated algae captured in spill tray 149 is then delivered by pumps to the downstream processing stage 70 where the algae cells are lysed and lipid oils removed and processed in manner known in the art of extraction of lipid oil from algae.

Injection of Carbon Dioxide and NOx into the “Multi-Panelled Sealed Tent(s)” 12

Carbon dioxide and NO_{X} gases 44 sourced from the algae separation system 137 are directed via a manifold to “multi-panelled sealed tent(s)” 12 using valve(s) 34 at or about the “far end” of the “multi-panelled sealed tent(s)” 12 and via the “far end” plate(s) 16.

The direction of carbon dioxide 43,44 flow from the “far end” plate 16 to the “near end” plate 17 (where it becomes enriched with oxygen 37), though preferred, is not limiting, as there may be occasions in which the direction of carbon dioxide 43,44 flow may need to be from “near end” plate 17 to “far end” plate 16.

### Processing of Algae

Harvesting and processing of algae to biofuel may generally conveniently proceed as described in the Applicant's co-pending International Application No. PCT/AU2022/051155 incorporated herein by reference and not repeated here. However, the flocculation of algae potentially avoids the need for power consuming centrifugal separation during harvesting prior to processing of algae to produce biofuel.

The key step in processing of algae is extraction of algal lipids or lipid oils which are then converted to biofuel. A potential benefit in providing algae with sufficient nitrogen (for example, 5% of dry matter) using the method and system as described above is that the remnant components of the algae cells, with lipid oils removed, have an increased measure of nitrogen by weight, and can serve as a nitrogen fertiliser for agriculture. For example, and only by way of illustration, if algae contain 5% nitrogen and 30% lipid oil, removal of the lipid oils will leave 7% by weight nitrogen in the remnant algae. This is not as bountiful as urea (a common fertiliser containing approximately 47% nitrogen) but it is a byproduct of the invention and the nitrogen is organically bound to the algae remnant as distinct to highly soluble urea (which has water runoff issues) and so is a more persistent nitrogen product which can be used for good purpose as a nitrogen fertiliser in the agricultural industry.

### EXAMPLE

A 44 ML/year of lipid oil extracted at a yield of 30% from algae infers that approximately 130,000 Tonne of algae is produced containing 5% Nitrogen, or 6,500 T Nitrogen which is 14,000 T urea or alternatively 7,800 T ammonia. In 2022 terms the cost of this fertiliser would otherwise be about USD14 million/year plus freight and taxes.

Application of 88,000 T Bagasse/yr at 40% moisture mixed with 30,000 T Coal/yr to produce the same 44 ML/year of lipid oil provides sufficient NO_{X} gases burnt at 1350 degree Centigrade and at 1.5 atm pressure in 75% Oxygen, 21.6% CO₂, 1% N₂ (air) and 2.4% NO (remnant from AGS) to produce 9,600 T nitric acid/year which can be neutralised after flocculation with 2,600 T ammonia, a 3 multiple in reduction in the otherwise above ammonia requirement.

2,600 T ammonia can be produced without the parasitic power requirement (or cost) that would otherwise be required to produce 7,800T ammonia to provide a healthy algae medium.

9,600 T nitric acid will provide a pH of about 2.8 in the algae separation system 137 which is enough acid to cause flocculation of algae, and thus eliminate the electrical power cost of centrifugal separation. This is another parasitic power saving achievable using the systems and methods of the present invention.

Furthermore, the algae itself produces 44 ML of lipids that can be hydro-processed into paraffin oils such as renewable diesel, which provides the potential for a substantial profit.

Algae concentration of 1.57 μm/L in algae medium with a flow rate through the algae separation system 137 of 225,000 kl/day would provide the 44 ML/year lipid oil outcome with a flocculation recovery of 75%. The algae separation system would therefore need to be suitably dimensioned providing approximately 50,000 kL algae medium storage to provide sufficient time latency to allow the nitrous acids to decompose into nitric acids which react readily with ammonia.

It will be appreciated by those skilled in the art that variations and modifications to the system for generating and using carbon dioxide and NOx described herein will be apparent without departing from the spirit and scope thereof. The variations and modifications as would be apparent to persons skilled in the art are deemed to fall within the broad scope and ambit of the invention as herein set forth.

1-20. (canceled)

21. A system for generating NOx gases for growth of algae comprising:
(a) a closed system combustion stage to combust carbonaceous material at a flame temperature and pressure promoting generation of an exhaust gas comprising a controlled proportion of carbon dioxide and a controlled pre-determined quantity of NOx gases as nutrients for algae;
(b) a closed system algae growth and oxygen generation stage for receiving the exhaust gas from the closed system combustion stage and delivering said exhaust gas as at least a portion of a feedstock gas for growing algae in an algae growth medium in which solubilised species of nitrogen including at least acid forming NOx and its reaction products with algae growth medium are metabolised by growing algae and directing an exit gas substantially comprising algal produced oxygen, carbon dioxide and non-solubilised NOx in controlled proportions from the closed system algae growth and oxygen generation stage to the closed system combustion stage, said exit gas comprising oxygen in major proportion and carbon dioxide and non-solubilised NOx in minor proportion;
(c) dissociating insoluble NOx received in the exit gas from the closed system algae growth and oxygen generation stage in the closed system combustion stage and reforming said NOx into the same or other NOx gas constituents which are contained within the exhaust gas; and
(d) a control system for controlling operation of the closed system combustion stage and closed system algae growth and oxygen generation stage,
wherein the control system controls pH of the algae growth medium within the closed system algae growth and oxygen generation stage to achieve a selected condition.

22. The system of claim 21, wherein non-solubilised NOx gases directed to the closed system combustion stage from the closed system algae growth and oxygen generation stage, are dissociated under high combustion temperatures and reformed into the same or other NOx gas constituents.

23. The system of claim 21, comprising a corona discharge device placed in the exhaust gas from the closed system combustion stage to transform NOx into nitrous and nitric acids.

24. The system of claim 22, wherein the control system controls pH of the algae growth medium by mixing the feedstock gas with at least one of an alkaline solution and alkaline producing gases to achieve a condition selected from the group consisting of flocculating algae and conditioning the pH of the algae growth medium including for growth of algae.

25. The system of claim 24, further comprising a gas production system for producing a gas or alkaline solution formed by dissolution of said gas in water to blend with said feedstock gas within the algae growth medium, said gas production system optionally being an ammonia gas or ammonia solution production system.

26. The system of claim 25, wherein the control system controls addition of ammonia gas or ammonia solution to the algae growth medium to buffer the pH of the algae growth medium.

27. The system of claim 26, wherein:
a. in a flocculation process requiring an acidic algae growth medium (as dependent on the algae species), ammonia is introduced separately to each space formed by baffles of a conditioning vessel; or
b. in a flocculation process requiring an alkaline algae growth medium (as dependent on the algae species) exhaust containing NOx and/or other acid forming gases from the closed system combustion stage forming nitrogen based acids is introduced separately to each space formed by baffles of the conditioning vessel
and dependent on pH as monitored in the space(s).

28. The system of claim 27, further comprising, within the conditioning vessel, reacting nitrogen containing acids derived from solubilised NOx with ammonia in the algae growth medium to produce in situ a soluble nitrogen fertiliser, said soluble nitrogen fertiliser optionally being ammonium nitrate.

29. The system of claim 23, wherein said closed system algae growth and oxygen generation stage is provided with storage for a gas mixture comprising carbon dioxide, oxygen and NOx to balance algae uptake of carbon dioxide and nitrogen containing constituents with carbon dioxide and NOx delivered by closed system combustion stage exhaust gases.

30. The system of claim 29, wherein composition of the exhaust gas and feedstock gas and consequential pH of the algae growth medium is controlled by controlling a plurality of the following:
(a) concentration of oxygen, CO₂, NOx gases and optionally N₂ directed to the closed system combustion stage;
(b) throughput rate of exit gas from the closed system algae growth and oxygen generation stage to the closed system combustion stage;
(c) combustion temperature;
(d) gas pressure during combustion;
(e) where introduced, throughput rate of nitrogen to the closed system combustion stage, nitrogen preferably being delivered in minor concentrations to the closed system combustion stage in the form of air;
(f) throughput rate of carbonaceous material for the closed system combustion stage; and
(g) quantities and pH of alkaline solution added by the control system.

31. The system of claim 30, further comprising an algae harvesting and processing system for processing algae lipids to a further chemical product, optionally a biofuel.

32. A method for generating NOx gases for growth of algae comprising:
(a) combusting carbonaceous material at a flame temperature and pressure promoting generation of an exhaust gas comprising a controlled proportion of carbon dioxide and a controlled pre-determined quantity of NO_{X} gases as nutrients for algae;
(b) receiving the exhaust gas from the closed system combustion stage in a closed system algae growth and oxygen generation stage as at least a portion of a feedstock gas for growing algae in an algae growth medium in which solubilised species of nitrogen including at least acid forming NOx and its reaction products with algae growth medium are metabolised by growing algae and directing an exit gas substantially comprising algal produced oxygen, carbon dioxide and non-solubilised NOx gases in controlled proportions from the closed system algae growth and oxygen generation stage to the closed system combustion stage, said exit gas comprising oxygen in major proportion and carbon dioxide and non-solubilised NOx in minor proportion; and
(c) dissociating under high combustion temperatures non-solubilised NOx gases in a closed system combustion stage, received from a closed system algae growth and oxygen generation stage and reforming into the same or other NO_{X} gas constituents; and
(d) controlling, by a control system, operation of the closed system combustion stage and closed system algae growth and oxygen generation stage,
wherein the control system controls pH of the algae growth medium to achieve a selected condition.

33. The method of claim 32, comprising controlling the pH of the algae growth medium by mixing the feedstock gas with at least one of an alkaline solution and alkaline producing gases to achieve a condition selected from the group consisting of flocculating algae and conditioning the pH of the algae growth medium.

34. The method of claim 33, wherein pH of the algae growth medium is controlled by the control system to flocculate algae, in a determined acidic range or in a determined alkaline range, with the preferred pH range influenced by the zeta potential of the growing algae species.

35. The method of claim 32, further comprising a gas production system for producing a gas or alkaline solution to blend with said feedstock gas within the algae growth medium to buffer pH of the algae growth medium, optionally an ammonia gas or ammonia solution production system.

36. The method of claim 35, further comprising reacting nitrogen containing acids with ammonia in the algae growth medium to produce in situ a soluble nitrogen fertiliser, optionally ammonium nitrate.

37. The method of claim 32, further comprising balancing algae uptake of carbon dioxide and NOx with rate of delivery of carbon dioxide and nitrogen containing constituents delivered by closed system combustion stage exhaust gases.

38. The method of claim 37, wherein composition of the exhaust gas and feedstock gas and consequential pH of the algae growth medium is controlled by controlling at least one of the following:
(a) concentration of oxygen, CO₂, NOx gases and optionally N₂ directed to the closed system combustion stage;
(b) throughput rate of exit gas from the closed system algae growth and oxygen generation stage;
(c) combustion temperature;
(d) gas pressure during combustion;
(e) where introduced, throughput rate of nitrogen to the closed system combustion stage, nitrogen preferably being delivered to the closed system combustion stage in minor concentrations in the form of air;
(f) throughput rate of combustible material for the closed system combustion stage; and
(g) quantities and pH of alkaline solution added by the control system.

39. The method of claim 38, further comprising processing algae lipids to a further chemical product in an algae harvesting and processing system.

40. The method of claim 38, further comprising implementing a corona discharge in the exhaust gas from the closed system combustion stage to transform NOx into nitrous and nitric acids.
